(19) United States
(12) Patent Application Publication
(10) Pub. No.: US 2011/0117583 A1
(43) Pub. Date: May 19, 2011

(54) METHOD FOR DETECTING THE EXISTENCE OF RENAL CALCULI AND/OR INFLAMMATION OF THE EXCRETORY URINARY TRACTS

(76) Inventor: Werner Hoffmann, Magdeburg (DE)

(21) Appl. No.: 12/991,344

(22) PCT Filed: Apr. 28, 2009
(86) PCT No.: PCT/DE09/00593

§ 371 Date: Jan. 7, 2011

(30) Foreign Application Priority Data
May 5, 2008 (DE) 10-2008-022-609.2

(51) Int. Cl. G01N 33/68

(52) U.S. Cl. 435/7.92; 436/501

(57) ABSTRACT

A non-invasive method for detecting disease of the excretory urinary tract and presence of renal calculi is provided. According to the method, the presence or concentration of one or more TFF peptides from among TFF1, TFF2, and TFF3 is determined in a urine sample, the presence or concentration being indicative of urinary tract disease or presence of renal calculi.

### BACKGROUND OF THE INVENTION

The invention relates to a method for detecting the existence of renal calculi and/or inflammations of the excretory urinary tracts, including the detection of specific diseases.

Different diseases of the excretory urinary tracts [e.g. renal calculi and urethral infections (UTI)] can only be diagnosed by relatively expensive methods. Frequently, the diagnosis is not secure (e.g. ultrasound, growing cultures). For calculi in particular, the diagnosis is often problematic and requires expensive devices and very experienced doctors.

DE 696 19 416 T2 discloses a method and reagents for the immunological detection of cortisol in urine.

DE 695 23 707 T2 describes a method for the detection of creatinine and of proteins contained in urine.

DE 44 44 533 A1 describes an apparatus and a method for performing a test to detect particles in urine (test for determining the susceptibility of a test subject for renal calculi).

DE 38 87 314 T2 provides a method for the detection of basic fetal proteins in urine and an analysis preparation suited for it.

DE 18 12 584 discloses a rapid and sensitive detection of bacteria in blood products, urine and other liquids.

DE 38 38 718 A1 shows a method for detecting diseases combined with metabolism abnormalities of L-fucose, such as stomach ulcers, liver cirrhosis and carcinomas in humans. Said method compares the concentrations of free L-fucose with their normal value in a sample taken from a subject.

The publication Williams, G. R.; Wright, N. A.: Trefoil Factor Family Domain Peptides, Virchows Archiv (1997), Vol. 431, No. 5, pages 299-304 discloses the prognostic importance of the pathologic expression of TFF peptides for different carcinomas, including cancers of the excretory urinary tracts (particularly of the cyst).

The publication Vestergaard, E. M.: Plasma Levels of Trefoil Factors are Increased in Patients with Advanced Prostate Cancer, Clinical Cancer Research (2006), Vol. 12, No. 3, pages 807-812 reveals an ELISA immunoassay for the detection of TFF in combination with the diagnosis of prostate cancer.

According to these two publications, the TFF detection is performed in cells withdrawn per biopsy or in the blood by means of conventional bioanalytical detection methods, i.e., an invasive procedure in which cells or blood of the affected patient are taken.

TFF (Trefoil Factor Family) peptides are a family of luminal protection peptides that, among other functions, play a key role for the protection, regeneration and repair of mucous epithelia.

The peptides TFF1, TFF2, and TFF3 are present in humans. The physiological presence of TFF1 (previous names: pS3, BCEI, pNR-2, pNR-105) and TFF2 is mainly restricted to the stomach whereas TFF3 (previous names: Intestinal Trefoil Factor/ITF, hP1.B) is synthesized by many mucous epithelia. TFF2 (previous names: pancreatic spasmolytic polypeptide/PSP, spasmolytic polypeptide/SP, human spasmolytic polypeptide/hSP) is a component of the gastric juice and therefore it appears both in a glycosilated and a non-glycosilated form.

Moreover, in contrast to TFF1, gastric TFF2 is bound to mucin (Kouznetsova et al. (2007) Cell. Physiol. Biochem. 20:899-908).

An overexpression of TFF3 is known to occur with prostate cancer. since normal human urine contains only a very low concentration of TFF peptides, neither TFF1, TFF2, nor TFF3 can be normally detected by Western blot analysis.

### SUMMARY OF THE INVENTION

The object of the present invention is to provide a method for detecting diseases of the excretory urinary tracts which can be carried out non-invasively, thus allowing first indications, such as the presence of renal calculi and/or inflammations (e.g. after urinary tract infections) in the excretory urinary tracts, to be rapidly and cost-effectively detected, in order to resolve rapidly and cost-effectively e.g. microhaematuria caused by inflammation.

It was found out that TFF2 and, to a lesser extent, TFF1 and TFF3 are present in the urine of patients suffering from renal calculi and in patients with UTI (urinary tract infections) in considerably higher concentrations than in healthy patients, and that the presence of TFF2 can be detected. The TFF2 concentration in UTI patients is higher than in patients with renal calculi, thus making differential diagnostics possible.

In this invention, a semi-quantitative detection of TFF2 is achieved by means of Western blot analyses of urine samples after acetone precipitation, and subsequent detection by using chemiluminescence (ECL system), and digital state-of-the-art analysis.

The presently disclosed method combines biochemical methods (see, for example, Kouznetsova et al. (2007) Cell. Physiol. Biochem. 20:899-908). The procedure ensures that only glycosilated TFF2 present in the urine of the patient is included in the analysis.

Apart from these highly specific quantitative detection methods for TFF2 in urine, other detection methods can also be applied in the inventive procedure, such as enzyme-linked immunosorbent assay (ELISA), stripe test, radioimmunoassay (RIA), immunoradiometric assay (IRMA), or different chromatographic methods. A relevant immunoassay for TFF2 is disclosed in Vestergaard et al. (2004) Scand. J Clin. Lab. Invest. 64:146-156).

The inventive biochemical method has the advantage that it is a cost-effective, rapid test. The results provided by the test provide a basis for making decisions about additional possible applications/examinations that are often expensive and stressful for the patient. This approach allows the economical use of the currently very limited financial resources in the health sector and avoids unnecessary examinations that are stressful for the patient.

### DETAILED DESCRIPTION OF THE INVENTION

In the following the invention is explained by three embodiments, without being limited to them.

### 1^{st} Embodiment

A determination of the TFF2 concentration in urine of patients with suspected UTI is described below.

4 volumes (e.g. 2000 μL) of cold acetone (−20° C.) is added to 1 volume (e.g. 500 μL) of a urine sample (e.g. morning midstream urine) of a patient with suspected UTI. The urine proteins are precipitated at −20° C. for at least 2 hours. The precipitated proteins are then removed by centrifugation at 4° C. and 12,000×g for 10 minutes. The supernatant is eliminated and the pellet is air-dried. Afterwards, the pellet is absorbed in 0.5% SDS (in half the original urine volume, e.g. in 250 μL).

Then, 5 μL 4-fold sample buffer is added to 15 μL of the thus prepared sample for the gel electrophoresis. The mixture is heated in a boiling water bath for 4 minutes and immediately treated by a SDS polyacrylamid gel ectrophoresis (SDS-PAGE; 15%). All steps indicated in the following are performed in accordance with Kouznetsova et al. (Kouznetsova I, Laubinger W, Kalbacher H, Kalinski T, Meyer F, Roessner A, Hoffmann W (2004), Biosynthesis of Gastrokine-2 in the Human Gastric Mucosa: Restricted Spatial Expression Along the Antral Gland Axis and Differential Interaction with TFF1, TFF2 and Mucins. Cell. Physiol. Biochem. 20:899-908).

After treating with SDS-PAGE, the proteins are transferred to a nitrocellulose membrane, fixed with 0.1% glutaraldehyde and incubated in PBS with an affinity-pured polyclonal antiserum against TFF2 (e.g. anti-hTFF2-1; Jagla W, Wiede A, Dietzmann K, Rutkowski K, Hoffmann W (2000), Co-Localization of TFF3 Peptide and Oxytocin in the Human Hypothalamus. FASEB J. 14:1126-1131). The bands containing TFF2 are visualized by a commercial ECL detection system available from GE Healthcare. The Western blot analysis image is stored electronically (GeneGnome system, syngens). GeneTools gel analysis software (syngens)is used for the semi-quantitative analysis of the bands. In this step, either the glycosilated or the non-glycosilated TFF2 forms can be analyzed separately.

The calibration/verification can be made via recombinant TFF2. Considerably increased TFF2 values compared with reference values of healthy persons are a sign of an inflammation such as caused by UTI.

### 2^{nd} Embodiment

A determination of the TFF2 concentration in urine of patients with microhaematuria is described below.

A 10 ml urine sample (e.g. morning midstream urine) of a patient with unclear microhaematuria is centrifuged at 5,000×g for 15 minutes and an aliquot of the supernatant is subject to a specific ELISA for the quantitative determination of the TFF2 concentration. In this process, appropriate dilutions are treated according to the protocol of Vestergaard et al. (Vestergaard E M, Brynskov J, Ejskjaer, Clausen J T, Thim L, Nexo E, Paulsen S S (2004) Immunoassays of Human Trefoil Factors 1 and 2: Measured on Serum from Patients with Inflammatory Bowel Disease. Scand J. Clin. Lab. Invest. 64:146-156).

TFF2 values that are considerably higher than the reference values of healthy patients are a sign of an inflammation in the area of the excretory urinary tracts, such as caused by UTI or renal calculi, and can account for the microhaematuria. If data from a larger patient group becomes available, a differential diagnosis for the differentiation of UTI or renal nuclei could be developed from the determination of TFF2 concentration.

### 3^{rd} Embodiment

A determination of the TFF2 concentration in urine of patients with nephrolithiasis after extracorporeal shock wave lithotripsy (ESWL) is described below.

Today, high-grade nephrolithiasis is treated by extracorporeal shock wave lithotripsy (ESWL). In this process, the nuclei are crushed and then flushed out. However, this method can also cause lesions of the kidney epithelium or of the excretory urinary tracts as well as temporary inflammations. Therefore, an increase of the TFF2 concentration is normally observed in the urine after an ESWL treatment. This increase reflects the level of the inflammation. Generally, the value reaches the basal range after a few days. However, sometimes complications caused by subsequent inflammations are described.

Repeatedly measuring the TFF2 level (for example by ELISA at an interval of two day; see 2^{nd} embodiment) provides for observation of the healing process after ESWL therapy, which provides for early detection and prevention.

All elements presented in the description and in the embodiments can be employed in the invention both as single elements or in any combination.

1. A method for detecting one of an inflammatory disease of the excretory urinary tract and the presence of renal calculi comprising determining the presence in urine of at least one TFF-peptide selected from TFF1, TFF2, and TFF3 by one of Western blot analysis, enzyme-linked immunosorbent assay (ELISA), stripe test, radioimmunoassay (RIA), and immunoradiometric assay (IRMA).

2. The method of claim 1, wherein the at least one TFF-peptide is TFF2.

3. The method of claim 1, wherein the at least one TFF-peptide is one of TFF1 and TFF3.

4. The method of claim 1, further comprising determining the concentration of at least one TFF peptide selected from TFF1, TFF2, and TFF3 present in urine by one of Western blot analysis, enzyme-linked immunosorbent assay (ELISA), stripe test, radioimmunoassay (RIA), and immunoradiometric assay (IRMA).

5. The method of claim 4, wherein the at least one TFF-peptide is TFF2.

6. The method of claim 4, wherein the at least one TFF-peptide is one or more of TFF1 and TFF3.
